(19) United States
(12) Patent Application Publication
(10) Pub. No.: US 2007/0055268 A1
(43) Pub. Date: Mar. 8, 2007

(54) Cutting blocks for a surgical procedure and methods for using cutting blocks

(75) Inventors: Michael Utz, Tuttlingen (DE); Thomas Hermle, Rottweil (DE); S. David Stulberg, Chicago, IL (US)

Correspondence Address: Lipsitz & McAllister, LLC, 755 MAIN STREET, MONROE, CT, 06468, US

(73) Assignee: AESCULAP AG & Co. KG, Tuttlingen (DE)

(21) Appl. No.: 11/206,683

(22) Filed: Aug. 17, 2005

(51) Int. Cl. A61F 5/00

(52) U.S. Cl. 606/87

(57) ABSTRACT

Two cutting blocks are provided for preparing a femur for total knee arthroplasty. A first cutting block comprises a block body positionable on a resected distal end of the femur. A first slot is provided in the block body for guiding a saw blade when making an anterior femoral cut. A second slot is provided for guiding a saw blade when making an anterior chamfer cut. A third slot is provided for guiding a saw blade when making a posterior chamfer cut. The second cutting block comprises a block body positionable on the resected distal end of the femur after making the anterior femoral cut, the anterior chamfer cut, and the posterior chamfer cut. A slot is provided in the block body of the second cutting block for guiding a saw blade when making a posterior femoral cut.

### BACKGROUND OF THE INVENTION

The present invention relates generally to total knee arthroplasty. More specifically, the present invention relates to cutting blocks for use in preparing the distal femur to accept a femoral component of a prosthetic knee joint during knee joint replacement surgery.

During knee joint replacement surgery, the distal femoral surface of a patient's knee is often replaced with a femoral component of a prosthetic knee joint in the form of a curved metallic prosthesis. Such a femoral component may have a generally smooth and continuous outer curvature that faces a corresponding tibial component which is attached to the patient's tibia. The inner surface (i.e., the side abutting the femur) of this type of femoral component of the prosthetic knee joint is typically provided with a number of intersecting flat surfaces. A common femoral component provides five intersecting flat surfaces on its inner surface. One of the flat surfaces is adapted to face the posterior surface of the femur. A second flat surface is adapted to engage the anterior cortical surface of the femur. A third flat surface is adapted to engage the distal end of the patient's femur. Additionally, a pair of flat chamfer surfaces form diagonally extending surfaces which form an interface between the distal surface and the respective anterior and posterior surfaces.

Accordingly, surgery to implant a prosthetic knee joint requires that the distal femur be prepared to receive the femoral component of the prosthetic knee joint. Preparation of the distal femur involves cutting the femur to establish accurately positioned flat surfaces against which the femoral component of the prosthetic knee joint can rest after implantation. For the common prior art femoral component mentioned above, five saw cuts must be made to the distal femur corresponding to the five flat surfaces on the inner surface of the femoral component in order to prepare the distal femur to accept the femoral component.

An initial cut is made to provide a flat surface at the distal end of the femur, commonly referred to as the “distal cut”. Once the distal cut is made, an “anterior femoral cut” is made to provide a flat surface on the anterior of the femur, a “posterior femoral cut” is made to provide a flat surface on the posterior of the distal femur, an “anterior chamfer cut” is made to provide a diagonally extending surface between the cut distal surface and the cut anterior surface, and a “posterior chamfer cut” is made to provide a diagonally extending surface between the cut distal surface and the cut posterior surface.

Various prior art cutting guides, typically referred to as cutting blocks, have been developed for assisting a surgeon in guiding a saw blade when making the five cuts to establish the desired flat surfaces on the distal femur. Typically, one cutting block may be positioned on the distal femur for making the distal cut. After making the distal cut, another block may be positioned on the cut distal surface for making the remaining four cuts. Such a cutting block is referred to as a 4-in-1 cutting block and has four guide slots for making the four remaining cuts.

The 4-in-1 cutting block is positioned and secured upon a flat transverse surface established initially on the distal femur by the distal cut in order to guide the saw blade during the execution the anterior femoral cut, the posterior femoral cut, the anterior chamfer cut and the posterior chamfer cut. Such a 4-in-1 cutting block is disclosed in, for example, U.S. Pat. No. 4,892,093 to Zarnowski (Zarnowski).

U.S. Pat. No. 5,417,694 to Marik (Marik) discloses a distal femoral cutting block. Marik also discussed several prior art patents that describe various types of prior art devices for use in preparing the distal femur to accept a prosthetic knee joint.

When using a typical prior art 4-in-1 cutting block of the type described in Zarnowski, it is necessary to make a relatively large incision in the area of the distal femur to allow for the positioning and securing of the cutting block and for carrying out the four saw cuts.

Techniques have been developed to reduce the size of the incision required when using a typical 4-in-1 cutting block, in an attempt to make the knee joint replacement surgery less invasive. However, when making a smaller incision, the available operation field also becomes smaller. In order to compensate for the smaller operating field resulting from a smaller incision, the knee must be bent several times over the course of the operation, in order to enable access to different portions of the femur in the small operation field, so that the five different cuts may be made. In particular, in order to make the posterior femoral cut (also referred to as the dorsal condylar cut) using a 4-in-1 cutting block, the patient's leg must be brought to approximately 90 degrees of flexion, since preparation of the dorsal condyles from the front of the femur (i.e., the posterior femoral cut) is otherwise prevented by the tibia.

These techniques are referred to as “moving window” techniques, since flexing the leg to different degrees enables access to different portions of the femur in the same operation field or window. Although such moving window techniques allow for a smaller surgical incision, various amounts of tension are placed on the soft tissue of the leg and knee when it is bent to different angular positions. Such tension may impart trauma to the soft tissue of the leg and knee, lengthening recovery time and possibly weakening the leg and/or implanted knee prosthetic.

It would therefor be advantageous to provide methods and apparatus that enable the use of a small incision and field of operation, while minimizing the amount of soft tissue trauma to the leg and knee during knee joint replacement surgery. It would be further advantageous to provide methods and apparatus that do not require bending of the leg to 90 degrees for making the posterior femoral cut, thereby reducing soft tissue trauma to the leg and knee. It would also be advantageous to reduce the amount of space taken up in the operation field by prior art 4-in-1 cutting blocks.

The methods, systems, and cutting blocks of the present invention provide the foregoing and other advantages.

### SUMMARY OF THE INVENTION

The present invention relates to cutting blocks for use in preparing the distal femur to accept a femoral component of a prosthetic knee joint in knee joint replacement surgery, and methods for using these cutting blocks.

The cutting blocks provided in accordance with the present invention are designed for use after the distal femur has been initially prepared by making a distal femur cut to provide a resected distal end of the femur.

In accordance with an example embodiment of the present invention, two cutting blocks are provided, which may be used together as a system for preparing a femur for total knee arthroplasty.

A first cutting block comprises a block body positionable on a resected distal end of the femur. A first slot, which passes through the block body, is provided for guiding a saw blade when making an anterior femoral cut. A second slot, which passes through the block body, is provided for guiding a saw blade when making an anterior chamfer cut. A third slot, which passes through the block body, is provided for guiding a saw blade when making a posterior chamfer cut. The second cutting block comprises a block body positionable on the resected distal end of the femur after making the anterior femoral cut, the anterior chamfer cut, and the posterior chamfer cut. A slot, passing through the block body of the second cutting block, is provided for guiding a saw blade when making a posterior femoral cut.

The block body of the first cutting block may be positionable on the resected distal end of the femur during approximately 45 degrees of knee flexion. The block body of the second cutting block may be positionable on the resected distal end of the femur during approximately 90 degrees of knee flexion.

In one example embodiment, the block body of the first cutting block may be adapted to abut against a planar surface resulting from resection of the distal femur. In such an example embodiment, the block body of the first cutting block may comprise a vertical surface for abutting the planar surface resulting from the resection of the distal femur. The second and third slots of the first cutting block may pass through the vertical surface. The block body may further comprise a top portion extending above the vertical surface, with the first slot passing through the top portion. The block body of the first cutting block, when positioned on the resected distal femur, may extend approximately from above a top portion of the resected distal femur to a point between a midpoint of the resected distal femur and a bottom portion of the resected distal femur.

In a further example embodiment, the block body of the second cutting block may be adapted to abut against four planar surfaces obtained from making the anterior femoral cut, the anterior chamfer cut, and the posterior chamfer cut at the resected distal end of the femur. In such an example embodiment, the block body of the second cutting block comprises an upward sloping surface for abutting the planar surface resulting from the anterior chamfer cut and a downward sloping surface for abutting the planar surface resulting from the posterior chamfer cut. The block body of the second cutting block may further comprise a vertical surface disposed between the upward and downward sloping surfaces for abutting the planar surface resulting from resection of the distal end of the femur and a horizontal surface extending from an upper end of the upward sloping surface for abutting the planar surface resulting from the anterior femoral cut.

At least one fixing means may be provided for fixing the first cutting block or the second cutting block in position on the resected distal end of the femur.

The present invention also includes methods for preparing a femur for total knee arthroplasty using the first and second cutting blocks described above. In an example embodiment of such a method, the distal end of the femur is resected and the first cutting block is positioned on the resected distal end of the femur. With the first cutting block in place, an anterior femoral saw cut may be made through a first guide slot in the first cutting block, an anterior chamfer saw cut may be made through a second guide slot in the first cutting block, and a posterior chamfer saw cut may be made through a third guide slot in the first cutting block. These cuts may be made in any order. After these cuts are completed, the first cutting block may be removed from the resected distal end of the femur. Next, the second cutting block is positioned on the resected distal end of the femur, and a posterior femoral saw cut is made through a guide slot in the second cutting block. Once this final saw cut is completed, the second cutting block may be removed from the resected distal end of the femur. The femoral component of the prosthetic knee joint may now be fixed to the distal femur.

The knee may be brought into approximately 45 degrees of flexion prior to the positioning of the first cutting block and into approximately 90 degrees of flexion prior to the positioning of the second cutting block.

The first and second cutting blocks may be fixed in position prior to making the saw cuts through the guide slots of the first and second cutting blocks.

The features of the first and second cutting blocks used with the above-described method correspond to the first and second cutting blocks of the system described above.

Further, the present invention also covers various embodiments of the first and second cutting blocks as described above.

### BRIEF DESCRIPTION OF THE DRAWINGS

The present invention will hereinafter be described in conjunction with the appended drawing figures, wherein like reference numerals denote like elements, and:

FIG. 1 shows a perspective view of an example embodiment of a first cutting block in accordance with the present invention;

FIG. 2 shows an example front view of the cutting block of FIG. 1;

FIG. 3 shows an example bottom view of the cutting block of FIG. 1;

FIG. 4 shows an example side view of the cutting block of FIG. 1;

FIG. 5 shows a perspective view of an example embodiment of a second cutting block in accordance with the present invention;

FIG. 6 shows an example front view of the cutting block of FIG. 5;

FIG. 7 shows an example bottom view of the cutting block of FIG. 5;

FIG. 8 shows an example side view of the cutting block of FIG. 5; and

FIG. 9 (FIGS. 9a-9h) shows an example embodiment of a method of preparing a distal femur to accept a femoral component of a prosthetic knee joint using the cutting blocks shown in FIGS. 1-8.

### DETAILED DESCRIPTION

The ensuing detailed description provides exemplary embodiments only, and is not intended to limit the scope, applicability, or configuration of the invention. Rather, the ensuing detailed description of the exemplary embodiments will provide those skilled in the art with an enabling description for implementing an embodiment of the invention. It should be understood that various changes may be made in the function and arrangement of elements without departing from the spirit and scope of the invention as set forth in the appended claims.

In accordance with an example embodiment of the present invention, a cutting block system for preparing a femur for total knee arthroplasty is provided. The system comprises two cutting blocks for use in preparing the distal femur to accept a femoral component of a prosthetic knee joint. The two cutting blocks are used in succession and enable completion of the knee arthroplasty using a smaller than normal surgical incision and resulting smaller than normal surgical window than that which can be achieved using a typical prior art 4-in-1 cutting block. Such results are achieved with the present invention by the reduction in size and the overall outer geometry of the two cutting blocks when each is compared to that of a typical 4-in-1 cutting block. In other words, by dividing the 4 saw cuts required after resection of the distal femur and making three of the cuts using one cutting block and the last of the cuts using a different cutting block, the present invention allows each of these two separate cutting blocks to be smaller than a typical 4-in-1 cutting block. Thus, with the present invention, the joint replacement can be carried out with a smaller surgical incision and in a smaller surgical field. Further, the cutting blocks of the present invention enable the surgery to proceed in this smaller surgical field without the need to flex the patient's leg to more than 45 degrees before the first cutting block is positioned, thereby minimizing soft tissue damage to the leg and knee joint.

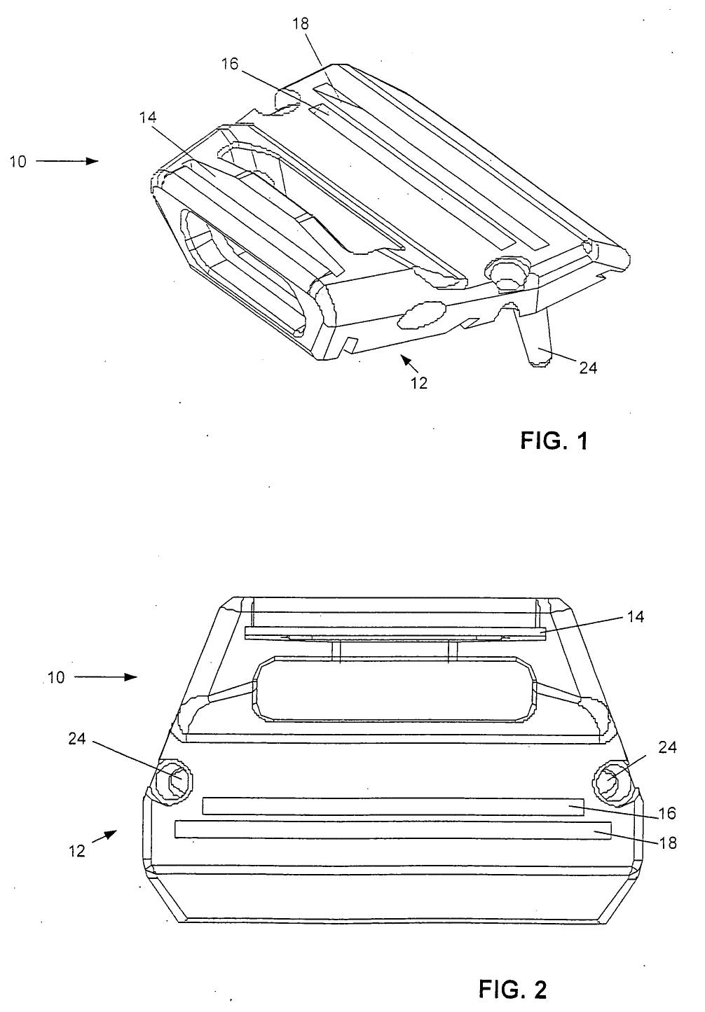

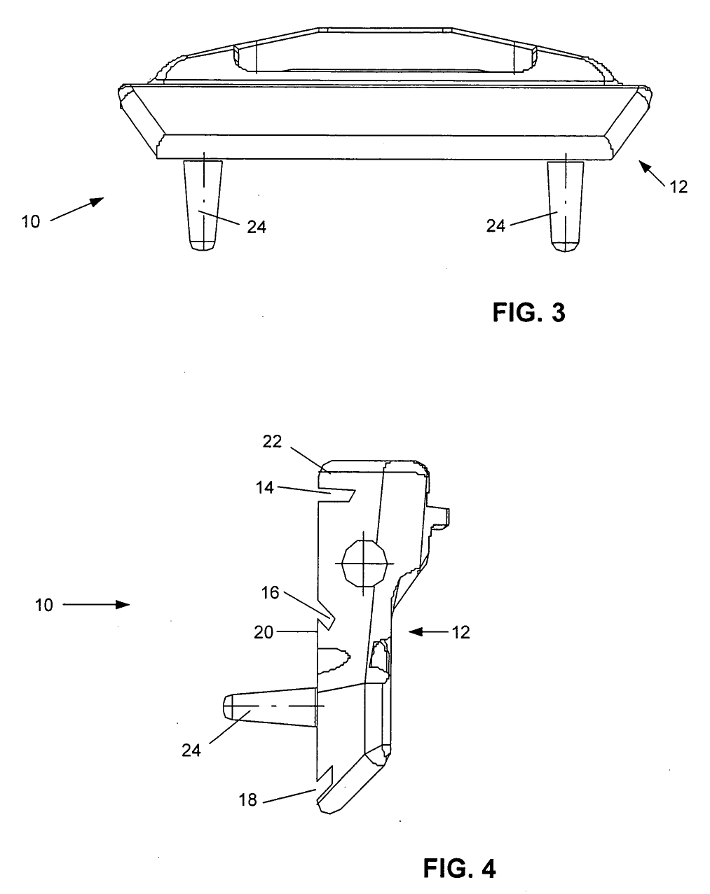

An example embodiment of a first cutting block 10 in accordance with the present invention is shown in FIGS. 1-4. FIG. 1 shows an example perspective view of the first cutting block 10, FIG. 2 shows an example front view of the first cutting block 10, FIG. 3 shows an example bottom view of the first cutting block 10, and FIG. 4 shows an example side view of the first cutting block 10.

The first cutting block 10 comprises a block body 12 positionable on a resected distal end of the femur. The first cutting block 10 may be considered to be a 3-in-1 cutting block, as it comprises three slots 14, 16, and 18 for making three of the 4 remaining cuts after resection of the distal femur. A first slot 14, which passes through the block body 12, is provided for guiding a saw blade when making an anterior femoral cut. A second slot 16, which passes through the block body 12, is provided for guiding a saw blade when making an anterior chamfer cut. A third slot 18, which passes through the block body 12, is provided for guiding a saw blade when making a posterior chamfer cut.

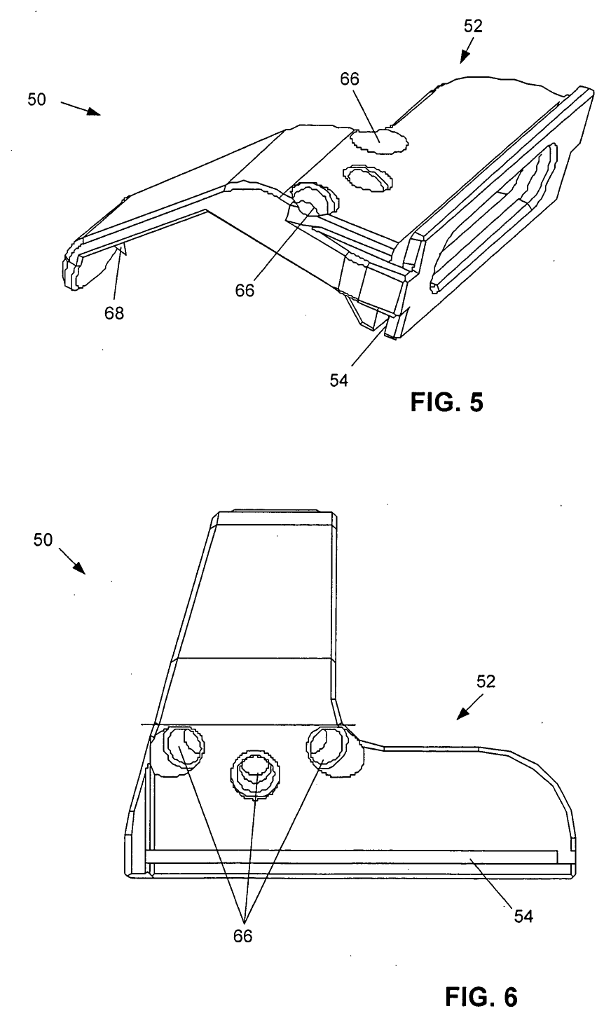

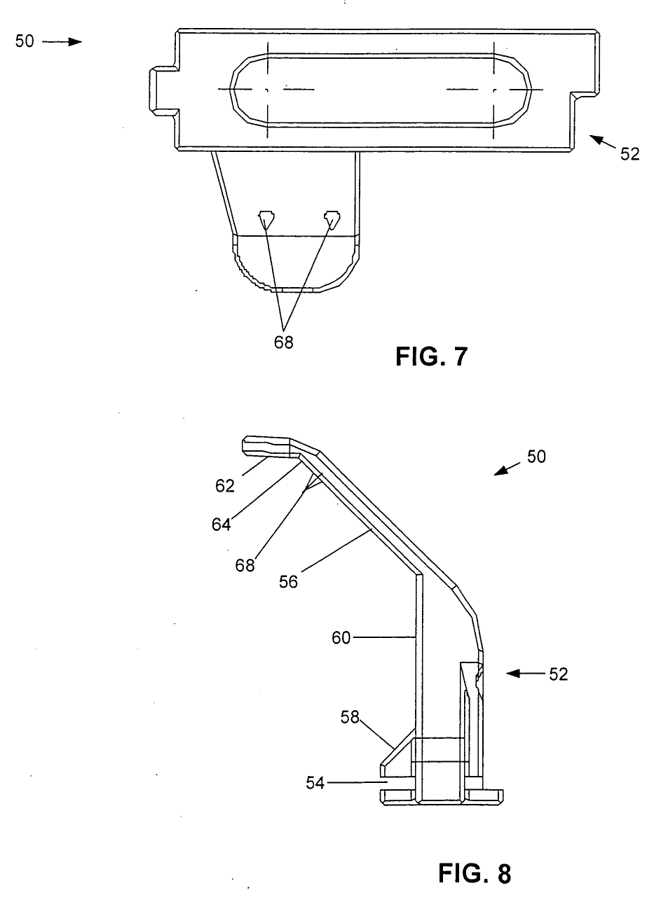

An example embodiment of a second cutting block 50 in accordance with the present invention is shown in FIGS. 5-8. FIG. 5 shows an example perspective view of the second cutting block 50, FIG. 6 shows an example front view of the second cutting block 20, FIG. 7 shows an example bottom view of the second cutting block 50, and FIG. 8 shows an example side view of the second cutting block 50.

The second cutting block 50 comprises a block body 52 positionable on the resected distal end of the femur after making the anterior femoral cut, the anterior chamfer cut, and the posterior chamfer cut using the first cutting block 10. A single slot 54, which passes through the block body 52 of the second cutting block, is provided for guiding a saw blade when making a posterior femoral cut.

The block body 12 of the first cutting block 10 may be positionable on the resected distal end of the femur during approximately 45 degrees of knee flexion. The block body 52 of the second cutting block 50 may be positionable on the resected distal end of the femur during approximately 90 degrees of knee flexion. Approximately 90 degrees of flexion is necessary to prevent the saw blade from contacting the tibia when making the posterior femoral cut, which would prevent the saw blade from moving freely. At approximately 90 degrees of flexion, the sawing plane for the posterior femoral cut is parallel to the upper side of the tibia and the saw blade will be able to move freely in the gap between the tibia and the femur.

In one example embodiment, the block body 12 of the first cutting block 10 may be adapted to abut against a planar surface resulting from resection of the distal femur. In such an example embodiment, the block body 12 of the first cutting block 10 may comprise a vertical surface 20 (FIG. 4) for abutting the planar surface resulting from the resection of the distal femur. The second and third slots 16 and 18 of the first cutting block 10 may pass through the block body 12 at an angle to this vertical surface 20. The block body 12 may further comprise a top portion 22 extending above the vertical surface 20, with the first slot 14 passing through the top portion 22. The block body 12 of the first cutting block 10, when positioned on the resected distal femur, may extend approximately from above a top portion of the resected distal femur to a point between a midpoint of the resected distal femur and a bottom portion of the resected distal femur.

In a further example embodiment, the block body 52 of the second cutting block 50 may be adapted to abut against four planar surfaces of the distal femur obtained from making the anterior femoral cut, the anterior chamfer cut, and the posterior chamfer cut at the resected distal end of the femur. In such an example embodiment, the block body 52 of the second cutting block 50 comprises an upward sloping surface 56 for abutting the planar surface resulting from the anterior chamfer cut and a downward sloping surface 58 for abutting the planar surface resulting from the posterior chamfer cut. The block body 52 of the second cutting block 50 may further comprise a vertical surface 60 disposed between the upward and downward sloping surfaces 56 and 58 for abutting the planar surface resulting from resection of the distal end of the femur and a horizontal surface 62 extending from an upper end 64 of the upward sloping surface 56 for abutting the planar surface resulting from the anterior femoral cut.

At least one fixing means may be provided for fixing the first cutting block 10 or the second cutting block 50 in position on the resected distal end of the femur. For example, as can be seen from FIGS. 2 and 3, hollow projections 24 extending from the block body 12 of the first cutting block 10 may be provided. These hollow projections extend within pre-drilled holes in the resected distal end of the femur and may be secured therein using bone screws or other suitable fixing means. Further, as can be seen from FIGS. 5 and 6, through holes 66 may be provided in the block body 52 of the second cutting block 50, via which the second block body 52 may be fixed to the resected distal end of the femur using bone screws or other suitable fixing means. In addition, as can be seen from FIGS. 7 and 8, one or more stabilizing pins 68 may be provided on the upward sloping surface 56 of the block body 52. These stabilizing pins may be pressed into the planar surface of the distal femur resulting from the anterior chamfer cut in order to maintain the block body 52 in position on the distal femur.

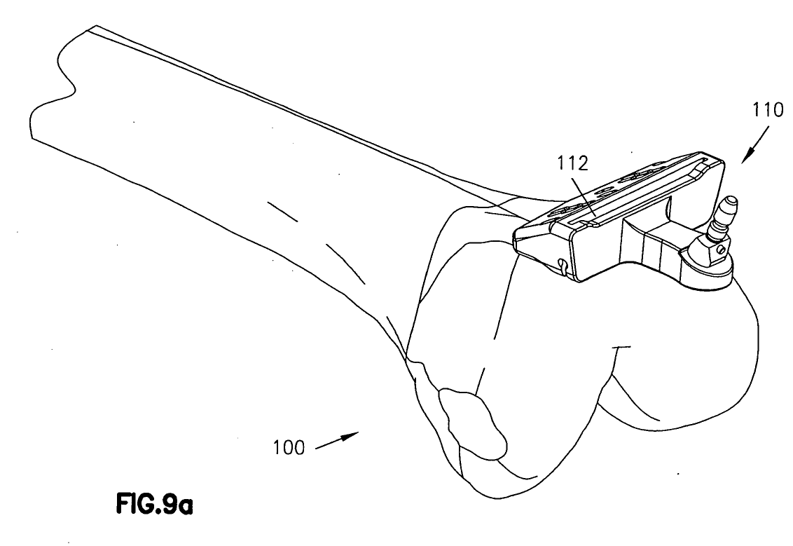

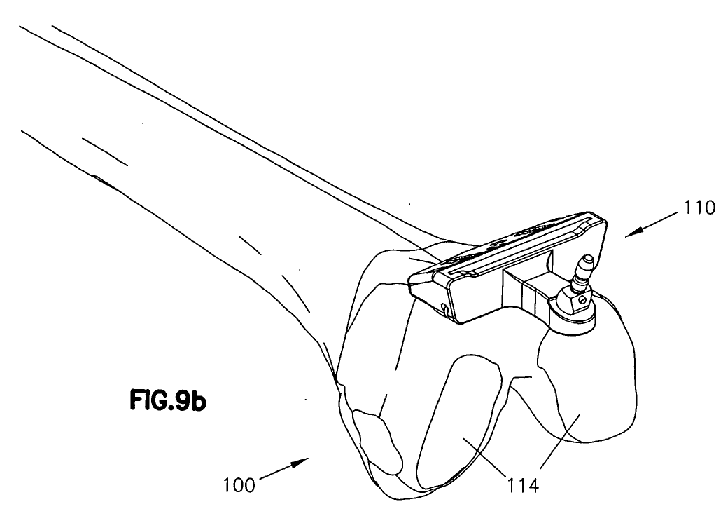

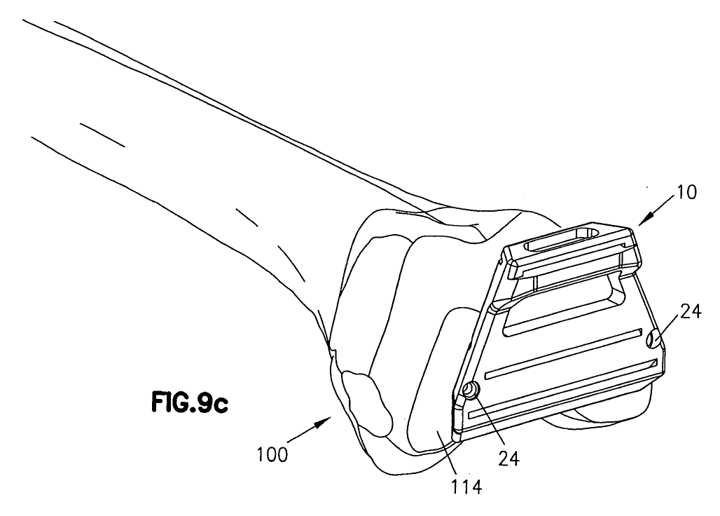

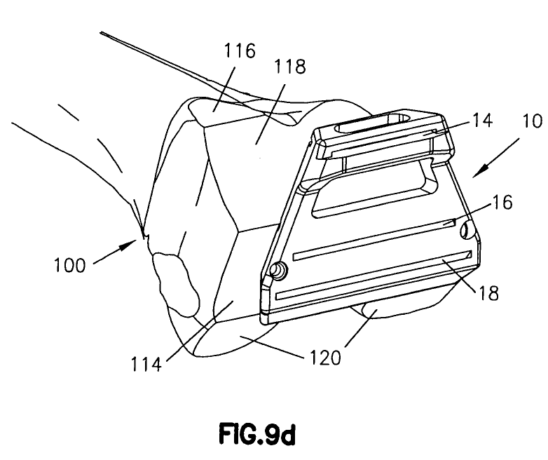

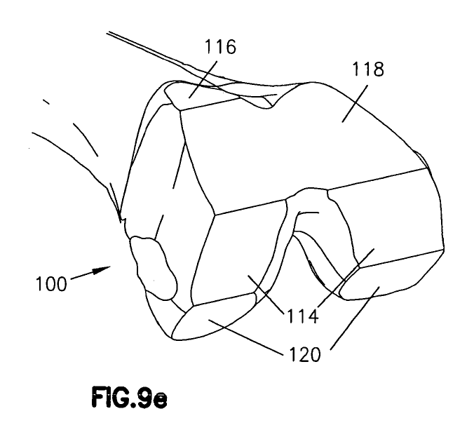

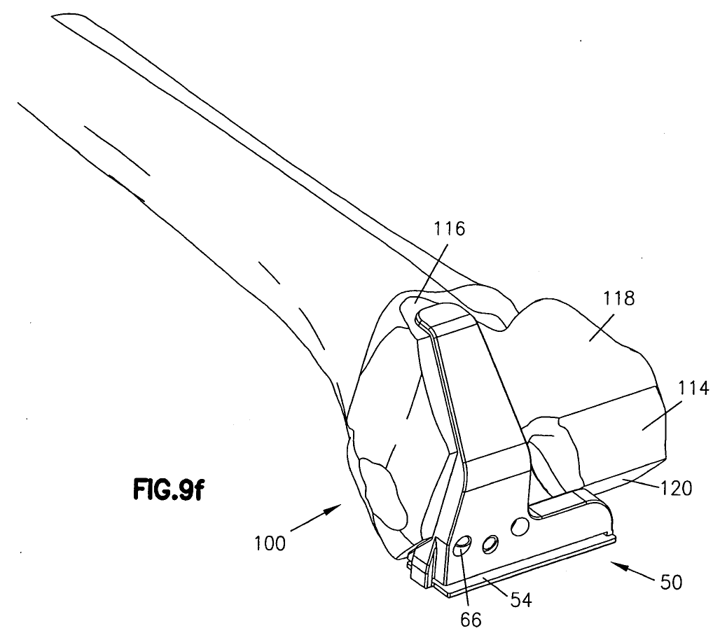

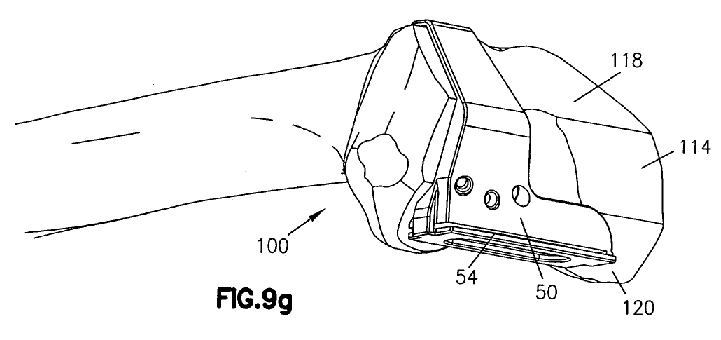

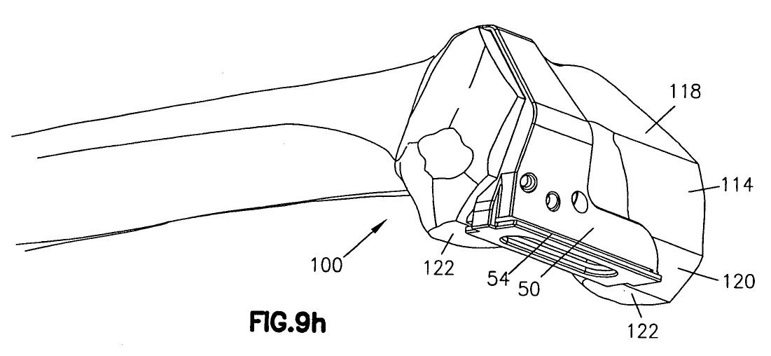

The present invention also includes methods for preparing a femur for total knee arthroplasty. An example embodiment of such a method is shown in FIGS. 9a-9h. FIG. 9a shows a distal femur cutting block 110 positioned on the distal end of the femur 100. The distal cutting block 110 may be positioned on the distal end of the femur 100 using a navigational aid as is known in the art. A guide slot 112 in distal cutting block 100 is used to guide a saw blade for making the distal cut at the distal end of the femur 100. The type of distal cutting block and its positioning is not material to the present invention. Those skilled in the art will appreciate that there are many available devices for use in resecting the distal end of the femur, and many ways in which such devices can be positioned on the distal femur.

FIG. 9b shows the resected distal end 114 of the femur 100 after the distal cut is made. The distal cutting block 110 can then be removed.

As shown in FIG. 9c, the first cutting block 10 (FIGS. 1-4) can be positioned on the resected distal end 114 of the femur 100 with the knee in approximately 45 degrees of flexion. The first cutting block may be positioned using a navigational aid as is known in the art. For example, the first cutting block may be positioned using the OrthoPilot Navigation System developed by Aesculap AG & Co. KG, the assignee of the present invention. For example, the first cutting block may be positioned using a femur orientation block and the OrthoPilot system. Two holes may be drilled through the orientation block (not shown), after which the orientation block can be removed. The two holes correspond to the locations of the hollow projections 24 on the first cutting block 10. The first cutting block 10 can then be fixed in position on the resected distal end 114 of the femur 100 by inserting the hollow projections 24 into the holes in the resected distal end of the femur. The first cutting block 10 can be secured to the distal end of the femur by, for example, bone screws screwed through the hollow projections 24 and into the distal end of the femur 100. The positioning of the first cutting block 10 is not the focus of the present invention, and the use of the OrthoPilot Navigation System and an orientation block is only one example of how the first cutting block 10 can be positioned. Those skilled in the art will appreciate that other types of navigation aids and positioning means can be used to position the first cutting block 10 on the resected distal end 114 of the femur 100. Further, those skilled in the art will appreciate that there may be several different options for fixing the first cutting block 10 to the resected distal end 114 of the femur 100.

As shown in FIG. 9d, with the first cutting block 10 in place on the resected distal end 114 of the femur 100, an anterior femoral saw cut 116 may be made through a first guide slot 14 in the first cutting block 10, an anterior chamfer saw cut 118 may be made through a second guide slot 16 in the first cutting block 10, and a posterior chamfer saw cut 120 may be made through a third guide slot 18 in the first cutting block 10. These three cuts may be made in any order. After these cuts are completed, the first cutting block 10 may be removed from the resected distal end 114 of the femur 100.

FIG. 9e shows the planar surfaces resulting from the distal cut 114, the anterior femoral saw cut 116, the anterior chamfer saw cut 118, and the posterior chamfer saw cut 120 after removal of the first cutting block 10.

Next, as shown in FIGS. 9f and 9g, the second cutting block 50 (FIGS. 5-8) is positioned on the resected distal end 114 of the femur 100. In order to position the second cutting block 50, the knee is brought into approximately 90 degrees of flexion. The second cutting block 50 can then be accommodated in the space made available after completion of the posterior chamfer saw cut 120, which creates a chamber for accommodating the body block 52 of the second cutting block 50. The second cutting block 50 may be positioned using a navigation aid, such as the OrthoPilot® Navigation System mentioned above or other means as will be apparent to those skilled in the art. The second cutting block 50 may be fixed to the distal end of the femur 100 via bone screws inserted into the distal femur through the through holes 66 and via stabilizing pins 68 as discussed above in connection with FIGS. 5-8.

As shown in FIG. 9h, a posterior femoral saw cut 122 is made through a guide slot 54 in the second cutting block 50. Once this final saw cut 122 is completed, the second cutting block 50 may be removed from the resected distal end 114 of the femur 100.

The distal end of the femur 100 is now prepared for accepting a femoral component of a prosthetic knee joint. Such a prosthesis may have a generally smooth and continuous outer curvature that faces a corresponding tibial component which is attached to the patient's tibia. The inner surface (i.e., the side abutting the femur) of such a femoral component of the prosthetic knee joint is provided with five intersecting flat surfaces which correspond to the planar surfaces resulting from the distal saw cut 114, the anterior femoral saw cut 116, the anterior chamfer saw cut 118, the posterior chamfer saw cut 120, and the posterior femoral saw cut 122. An example of such a prosthetic knee joint is the e.motion® implant developed by Aesculap AG & Co. KG, the assignee of the present invention.

As discussed above, the shape of the cutting blocks enable the first cutting block 10 to be positioned and the saw cuts to be made through the first cutting block 10 with the knee in approximately 45 degrees of flexion or less. The second cutting block 50 may be positioned and the saw cuts may be made through the second cutting block 50 with the knee in approximately 90 degrees of flexion. Since the first four saw cuts have been made prior to positioning of the second cutting block 50 on the resected distal end of the femur 114, there is ample room in the knee joint to enable 90 degrees of flexion without increasing tension on the soft tissue of the knee joint.

It should now be appreciated that the present invention provides advantageous systems, methods and cutting blocks for preparing the distal end of a femur to accept a femoral component of a prosthetic knee joint. By using two separate cutting blocks for the four cuts made after the distal cut, the present invention enables total knee arthroplasty using a smaller surgical incision and resulting smaller field of operation. Further, the present invention minimizes soft tissue damage in and around the leg and knee area due to a reduction in tension on the soft tissue of the knee joint during the surgery.

Although the invention has been described in connection with various illustrated embodiments, numerous modifications and adaptations may be made thereto without departing from the spirit and scope of the invention as set forth in the claims.

What is claimed is:

1. A cutting block system for preparing a femur for total knee arthroplasty, said system comprising:
a first cutting block, comprising:
a block body positionable on a resected distal end of said femur;
a first slot passing through said block body for guiding a saw blade when making an anterior femoral cut;
a second slot passing through said block body for guiding a saw blade when making an anterior chamfer cut; and
a third slot passing through said block body for guiding a saw blade when making a posterior chamfer cut; and
a second cutting block, comprising:
a block body positionable on said resected distal end of said femur after making said anterior femoral cut, said anterior chamfer cut, and said posterior chamfer cut; and
a slot passing through said block body for guiding a saw blade when making a posterior femoral cut.

2. A cutting block system in accordance with claim 1, wherein:
said block body of said first cutting block is positionable on said resected distal end of said femur during approximately 45 degrees of knee flexion.

3. A cutting block system in accordance with claim 1, wherein:
said block body of said second cutting block is positionable on said resected distal end of said femur during approximately 90 degrees of knee flexion.

4. A cutting block system in accordance with claim 1, wherein:
said block body of said first cutting block is adapted to abut against a planar surface resulting from resection of the distal femur.

5. A cutting block system in accordance with claim 4, wherein said block body of said first cutting block comprises:
a vertical surface for abutting the planar surface resulting from said resection of said distal femur, said second and third slots passing through said vertical surface; and
a top portion extending above the vertical surface, said first slot passing through said top portion;
said block body of said first cutting block, when positioned on said resected distal femur, extending approximately from above a top portion of the resected distal femur to a point between a midpoint of the resected distal femur and a bottom portion of said resected distal femur.

6. A cutting block in accordance with claim 1, wherein:
said body block of said second cutting block is adapted to abut against at least the two planar surfaces obtained from making said anterior chamfer cut and said posterior chamfer cut at said resected distal end of said femur.

7. A cutting block system in accordance with claim 1, wherein:
said block body of said second cutting block is adapted to abut against four planar surfaces obtained from making said anterior femoral cut, said anterior chamfer cut, and said posterior chamfer cut at said resected distal end of said femur.

8. A cutting block system in accordance with claim 7, wherein said block body of said second cutting block comprises:
an upward sloping surface for abutting the planar surface resulting from said anterior chamfer cut;
a downward sloping surface for abutting the planar surface resulting from said posterior chamfer cut; and
a vertical surface disposed between the upward and downward sloping surfaces for abutting the planar surface resulting from resection of the distal end of the femur.

9. A cutting block system in accordance with claim 8, wherein:
said body block of said second cutting block further comprises a horizontal surface extending from an upper end of the upward sloping surface for abutting the planar surface resulting from the anterior femoral cut.

10. A cutting block system in accordance with claim 1, further comprising:
at least one fixing means for fixing said first cutting block or said second cutting block in position on said resected distal end of said femur.

11. A method for preparing a femur for total knee arthroplasty, comprising:
resecting a distal end of said femur;
positioning a first cutting block on the resected distal end of said femur;
making an anterior femoral saw cut through a first guide slot in said first cutting block;
making an anterior chamfer saw cut through a second guide slot in said first cutting block;
making a posterior chamfer saw cut through a third guide slot in said first cutting block;
removing said first cutting block from said resected distal end of said femur;
positioning a second cutting block on said resected distal end of said femur;
making a posterior femoral saw cut through a guide slot in said second cutting block; and
removing said second cutting block from said resected distal end of said femur.

12. A method in accordance with claim 11, further comprising:
bringing said knee into approximately 45 degrees of flexion prior to the positioning of the first cutting block.

13. A method in accordance with claim 11, further comprising:
bringing said knee into approximately 90 degrees of flexion prior to the positioning of the second cutting block.

14. A method in accordance with claim 11, wherein:
said first cutting block is adapted to abut against a planar surface resulting from resection of the distal femur.

15. A method in accordance with claim 14, wherein said first cutting block comprises:
a vertical surface for abutting the planar surface resulting from said resection of said distal femur, said second and third slots passing through said vertical surface; and
a top portion extending above the vertical surface, said first slot passing through said top portion;
said first cutting block, when positioned on said resected distal femur, extending approximately from above a top portion of the resected distal femur to a point between a midpoint of the resected distal femur and a bottom portion of said resected distal femur.

16. A method in accordance with claim 11, wherein:
said second cutting block is adapted to abut against at least the two planar surfaces obtained from making said anterior chamfer cut and said posterior chamfer cut at said resected distal end of said femur.

17. A method in accordance with claim 11, wherein:
said second cutting block is adapted to abut against four planar surfaces obtained from making said anterior femoral cut, said anterior chamfer cut, and said posterior chamfer cut at said resected distal end of said femur.

18. A method in accordance with claim 17, wherein said second cutting block comprises:
an upward sloping surface for abutting the planar surface resulting from the anterior chamfer cut;
a downward sloping surface for abutting the planar surface resulting from said posterior chamfer cut; and
a vertical surface disposed between the upward and downward sloping surfaces for abutting the planar surface resulting from resection of the distal end of the femur.

19. A method in accordance with claim 18, wherein:
said second cutting block further comprises a horizontal surface extending from an upper end of the upward sloping surface for abutting the planar surface resulting from the anterior femoral cut.

20. A method in accordance with claim 11, further comprising:
fixing said first cutting block in position prior to making said saw cuts through said guide slots of said first cutting block; and
fixing said second cutting block in position prior to making said saw cut through said guide slot of said second cutting block

21. A cutting block for preparing a femur for total knee arthroplasty, said cutting block comprising:
a block body positionable on a resected distal end of said femur;
a first slot passing through said block body for guiding a saw blade when making an anterior femoral cut;
a second slot passing through said block body for guiding a saw blade when making an anterior chamfer cut; and
a third slot passing through said block body for guiding a saw blade when making a posterior chamfer cut.

22. A cutting block in accordance with claim 21, wherein:
said block body is positionable on said resected distal end of said femur during approximately 45 degrees of knee flexion.

23. A cutting block in accordance with claim 21, wherein:
said block body is adapted to abut against a planar surface resulting from resection of the distal femur.

24. A cutting block in accordance with claim 23, wherein said block body comprises:
a vertical surface for abutting the planar surface resulting from said resection of said distal femur, said second and third slots passing through said vertical surface; and
a top portion extending above the vertical surface, said first slot passing through said top portion;
said block body, when positioned on said resected distal femur, extending approximately from above a top portion of the resected distal femur to a point between a midpoint of the resected distal femur and a bottom portion of said resected distal femur.

25. A cutting block for preparing a femur for total knee arthroplasty, said cutting block comprising:
a block body positionable on a resected distal end of said femur after making an anterior femoral cut, an anterior chamfer cut, and a posterior chamfer cut; and
a slot passing through said block body for guiding a saw blade when making a posterior femoral cut.

26. A cutting block in accordance with claim 25, wherein:
said block body is positionable on said resected distal end of said femur during approximately 90 degrees of knee flexion.

27. A method in accordance with claim 25, wherein:
said second cutting block is adapted to abut against at least the two planar surfaces obtained from making said anterior chamfer cut and said posterior chamfer cut at said resected distal end of said femur.

28. A cutting block in accordance with claim 25, wherein:
said block body is adapted to abut against four planar surfaces obtained from making said anterior femoral cut, said anterior chamfer cut, and said posterior chamfer cut at said resected distal end of said femur.

29. A cutting block in accordance with claim 28, wherein said block body comprises:
an upward sloping surface for abutting the planar surface resulting from the anterior chamfer cut;
a downward sloping surface for abutting the planar surface resulting from said posterior chamfer cut; and
a vertical surface disposed between the upward and downward sloping surfaces for abutting the planar surface resulting from resection of the distal end of the femur.

30. A method in accordance with claim 29, wherein:
said second cutting block further comprises a horizontal surface extending from an upper end of the upward sloping surface for abutting the planar surface resulting from the anterior femoral cut.
